(19) United States
(12) Patent Application Publication
(10) Pub. No.: US 2003/0069458 A1
(43) Pub. Date: Apr. 10, 2003

(54) Selective catalytic gas-phase hydrogenation of alkynes, dienes, alkenynes and/or polyenes

(76) Inventors: Thomas Hill, Mannheim (DE); Mathias Haake, Mannheim (DE); Ekkehard Schwab, Neustadt (DE); Andrea Frenzel, Edingen-Neckarhausen (DE); Helmut Worz, Mannheim (DE)

Correspondence Address: Herbert B. Keil, KEIL & WEINKAUF, 1350 Connecticut Ave., N.W., Washington, DC, 20036, US

(21) Appl. No.: 10/262,930

(22) Filed: Oct. 3, 2002

(30) Foreign Application Priority Data
Oct. 9, 2001 (DE) 10149631.1

(51) Int. Cl. C07C 5/00; C07C 5/03; C07C 5/08

(52) U.S. Cl. 585/265; 585/258; 585/259

(57) ABSTRACT

Alkynes, dienes, alkenynes and/or polyenes in an olefin-containing hydrocarbon stream are selectively hydrogenated catalytically in the gas phase in at least two reaction zones connected in series without introduction of part of this hydrocarbon stream between the penultimate reaction zone and the last reaction zone, wherein the hydrogen content in the reaction gas mixture upstream of the penultimate reaction zone and the degree of conversion in the penultimate reaction zone are set so that the reaction gas mixture contains at least 0.7% by volume of hydrogen at the outlet of the penultimate reaction zone.

The present invention relates to a process for the selective catalytic gas-phase hydrogenation of alkynes, dienes, alkenynes and/or polyenes in an olefin-containing hydrocarbon stream.

In refineries and petrochemical plants, large quantities of hydrocarbon streams are produced, stored and processed. Highly unsaturated compounds are frequently present in the hydrocarbon streams and their presence is known to lead to problems, particularly in processing and/or storage, or they are not the desired product and are therefore undesirable components of the corresponding hydrocarbon streams. These highly unsaturated compounds are alkynes, dienes, alkenynes and/or polyenes which are generally higher unsaturated homologues of the desired product present in the hydrocarbon stream concerned, which is usually a monounsaturated olefin or 1,3-butadiene. For example, in C₂ streams from steam crackers, the secondary component ethyne (trivial name “acetylene”) is undesired and ethene (trivial name “ethylene”) is the desired product, in C₃ streams, the secondary components propyne and propadiene (trivial names “methylacetylene” and “allene”, respectively) are undesired and propene (trivial name “propylene”) is the desired product, and in C₄ streams, the secondary components 1-butyne, 2-butyne, but-3-en-1-yne (trivial name “vinylacetylene”), 1,2-butadiene and butatriene are undesirable when 1,3-butadiene is to be isolated as desired product and processed further, and the secondary components mentioned plus 1,3-butadiene are undesirable in cases in which 1-butene or 2-butene (in the cis and/or trans form) are the desired products. Analogous problems occur in the case of hydrocarbon streams coming from an FCC plant or a reformer rather than a steam cracker.

Processes for reducing the concentration of alkynes, dienes, alkenynes and/or polyenes in an olefin-containing hydrocarbon stream have to meet a number of requirements. Thus, for example, the acetylene present in the C₂ stream from a steam cracker interferes in the polymerization of ethylene, so that the acetylene content of the C₂ stream, which is typically from 0.3 to 0.8% by volume, has to be reduced to values below 1 ppm by volume. Propyne and propadiene in the C₃ stream from a steam cracker, which are typically present in an amount of 2-3% by volume each, usually have to be removed from the C₃ stream down to a residual content of not more than 20 ppm by volume for chemical applications or not more than 5 ppm by volume for polymer applications. In the case of a C₄-hydrocarbon stream from a steam cracker, the objective in respect of C₄-alkynes or C₄-alkenynes is similar to that for a C₃ stream when 1,3-butadiene is to be extracted as desired product from the hydrocarbon stream, or in respect of the maximum residual content of 1,3-butadiene permissible for further processing in the case of a hydrocarbon stream which has already been freed of 1,3-butadiene. However, if 1- or 2-butene is the desired product, it is not only necessary to remove the alkynes, alkynenes and other dienes and polyenes but also to reduce the concentration of 1,3-butadiene, which is typically present in an amount of from 30 to 50% by volume in the C₄ stream, to a residual content of not more than 10 ppm by volume.

Alkynes, dienes, alkenynes and/or polyenes are customarily removed from an olefin-containing hydrocarbon stream by selective catalytic hydrogenation. For this purpose, C₂ streams are generally subjected to a gas-phase hydrogenation, while a liquid-phase hydrogenation is generally employed for C₅- and higher hydrocarbon streams and both gas-phase and liquid-phase processes are known for C₃ and C₄ streams. Catalysts used are customarily supported catalysts comprising noble metals, nowadays usually palladium catalysts or silver-doped palladium catalysts. Depending on the concentrations of the compounds to be removed from the hydrocarbon stream to be treated and their maximum permissible concentration, the hydrogenation is carried out in only one reactor or, more frequently, in a plurality of reactors connected in series. In the latter case, 2 or 3 reactors are usually used, with a degree of conversion of usually from 60 to 70% being set in the first reactor, a degree of conversion of from 30 to 40% being set in the second reactor and the remaining conversion down to the lower ppm region being set in the last reactor, if present. Although the use of 4 or more reactors is possible, it is usually disadvantageous for economic reasons. An overview of the work-up of hydrocarbon streams from a steam cracker is given, for example, by A. Watson in The Oil and Gas Journal, Nov. 8, 1976, pp. 179-182.

In such hydrogenation processes, two factors have to be taken into account. Firstly, the desired product such as ethylene, propylene, 1,3-butadiene or 1- or 2-butene is in each case also an unsaturated compound which can be hydrogenated over the catalyst, which leads to a loss of the desired product and therefore necessitates a very selective and carefully controlled hydrogenation of the undesirable compounds so as to form the desired olefinic homologues rather than the alkane from the more highly unsaturated impurities, or at least not to suffer any net loss of desired product by hydrogenation to the alkane. Secondly, the undesirable alkynes, dienes and polyenes are polymerized over the catalyst to form green oil, namely a mixture of various oligomers and polymers, which deposits on the catalyst, in the reactor and in downstream components of the plant and thus shortens the operating life of the catalyst and the intervals between necessary maintenance work, so that very rapid and complete hydrogenation of these undesirable green oil-forming components is required.

Various processes for the selective catalytic gas-phase hydrogenation of alkynes, dienes, alkenynes and/or polyenes in an olefin-containing hydrocarbon stream are known. Thus, DE-A-28 54 698 describes a multistage hydrogenation process in which the entire amount of hydrogen is introduced at the beginning into the first reaction zone and the hydrocarbon stream to be treated is divided and the individual substreams are fed in upstream of each of the individual reaction zones. This process is thus carried out using a considerable excess of hydrogen, based on the proportion of impurity to be hydrogenated, which largely avoids green oil formation but leads to comparatively high losses of desired product. This process is therefore not customarily employed.

EP-A-87 980 discloses the far more widely used process, namely the multistage hydrogenation of a hydrocarbon stream, in which hydrogen is introduced between each of the individual reaction zones. In this way, the amount of hydrogen available in each reaction zone precisely matches the amount necessary for the hydrogenation of the undesirable compounds. The aim of this is to prevent green oil formation to a sufficient extent while at the same time keeping the loss in yield caused by hydrogenation of desired product or overhydrogenation of the undesirable compound at a low level. Watson, loc. cit., teaches a variant of this process, namely the use of an amount of hydrogen which leads to a minimal excess of hydrogen at the outlet of the individual reactors. Further measures for optimizing such hydrogenation processes include, for example, careful temperature control, as disclosed in U.S. Pat. No. 4,707,245. A long-known method of increasing the selectivity of the catalyst in such processes is the addition of carbon monoxide as moderator, which makes the catalyst more selective but less active; however, this has the disadvantages that this carbon monoxide has to be separated off again and that the lower activity of the catalyst has to be compensated by a higher operating temperature, which favors green oil formation. The demand for a very low loss of desired product also necessitates the development and use of highly selective catalysts as are described, for example, in EP-A-992 284 and the literature cited therein. Also known is the use of structured catalysts, monoliths or catalyst packing, as are disclosed, for example, in U.S. Pat. No. 5,866,734 or in EP-A-965 384, in place of the widespread particulate catalysts.

In view of the ever higher demands made of the purity of olefins, the desire for a very low loss of desired product and the desire for long operating lives of catalysts and long maintenance intervals for plants, it is an object of the invention to find a process for removing alkynes, dienes and/or polyenes from olefin-containing hydrocarbon streams which firstly effectively prevents the formation of green oil and secondly leads to the desired product in high selectivity.

We have found that this object is achieved by a process for the selective catalytic gas-phase hydrogenation of alkynes, dienes, alkenynes and/or polyenes in an olefin-containing hydrocarbon stream in at least two reaction zones connected in series without introduction of part of this hydrocarbon stream between the penultimate reaction zone and the last reaction zone, wherein the hydrogen content in the reaction gas mixture upstream of the penultimate reaction zone and the degree of conversion in the penultimate reaction zone are set so that the reaction gas mixture contains at least 0.7% by volume of hydrogen at the outlet of the penultimate reaction zone.

The process of the present invention substantially suppresses green oil formation, while at the same time the loss of desired product is minimized or no such loss occurs at all. Surprisingly, despite the comparatively high excess of hydrogen employed, no increase in the overhydrogenation to alkanes is observed in the process of the present invention.

In the process of the present invention, alkynes, dienes, alkenynes and/or polyenes in an olefin-containing hydrocarbon stream are selectively hydrogenated catalytically and in the gas phase. In particular, the process of the present invention is used to hydrogenate acetylene in an ethylene-containing C₂ stream, to hydrogenate propyne and propadiene in a propylene-containing C₃ stream or to hydrogenate 1-butyne, 2-butyne, but-3-en-1-yne, 1,2-butadiene, butatriene and/or 1,3-butadiene in a 1,3-butadiene- and/or 1-butene- or 2-butene-containing hydrocarbon stream.

The process of the present invention is carried out in at least two reaction zones connected in series, preferably in two or three reaction zones connected in series. It is likewise possible to use four or more reaction zones connected in series, but this embodiment is usually disadvantageous for economic reasons, unless the alkynes, dienes, alkenynes and/or polyenes to be removed are present in an unusually large amount in the hydrocarbon stream. For the purposes of the present invention, a “reaction zone” is an individual reactor or an individual section of a reactor in which a plurality of reaction zones (for example individual, physically separate catalyst beds) are accommodated in a common reactor jacket. If reaction zones are operated adiabatically, cooling facilities are provided downstream of the adiabatically operated reaction zone to remove at least part of the heat of reaction evolved from the product stream, or other known cooling measures are employed, for example the circulation of part of the product from one reaction zone to the beginning of this reaction zone after this circulating gas stream has been cooled. As an alternative, the individual reaction zones can also be operated isothermally, i.e. with cooling facilities in the catalyst bed itself. The cooling facilities are matched to the quantity of heat evolved in the selective hydrogenation of the given hydrocarbon stream and can, if this quantity of heat is sufficiently low or a correspondingly hotter product stream is desired, also be omitted; this is part of a customary reactor and process design.

Furthermore, facilities for the introduction of hydrogen into the reaction gas mixture are provided upstream of the first reaction zone and upstream of the penultimate reaction zone. Preference is given to providing a facility for the introduction of hydrogen into the reaction gas mixture upstream of each reaction zone.

The hydrocarbon stream to be treated generally passes through the individual reaction zones in succession. It is possible but not necessary to divide this hydrocarbon stream into individual substreams and to introduce each of these substreams, apart from the substream fed into the first reaction zone, between two reaction zones. In particular, the introduction of part of the hydrocarbon stream to be treated between the penultimate reaction zone and the last reaction zone is not necessary. However, it is possible to introduce different hydrocarbon streams between the individual reaction zones as a function of their respective content of alkynes, dienes, alkenynes and/or polyenes. If, for example, a first hydrocarbon stream having a relatively high proportion of alkynes, dienes, alkenynes and/or polyenes and at the same time an otherwise comparable second hydrocarbon stream having a lower content of these compounds are to be hydrogenated selectively in a given petrochemicals or refinery complex, the first stream is, according to the present invention, hydrogenated in a plurality of reaction zones and the second stream is introduced between two reaction zones at a point at which the originally higher content of the compounds to be hydrogenated in the first hydrocarbon stream has already been reduced appropriately.

A substream of the product from a reaction zone can be taken from the product gas stream and reintroduced into the gas stream to be hydrogenated upstream of this reaction zone (it is in principle also possible to feed it in upstream of another reaction zone). This “circulating gas mode” is a frequently employed measure in such hydrogenations and serves, in particular, to set a sufficient conversion in a particular reaction zone, with the circulating gas also being able to be cooled before it is recirculated, so that the desired conversion is achieved without the product being heated undesirably by the heat of reaction liberated. Typical recycle ratios (recirculated substream to substream introduced for the first time into the reaction zone concerned) are in the range from 0 to 30.

The conditions set in the individual reaction zones correspond, with the exception of the excess of hydrogen to be set according to the present invention at the outlet of the penultimate reaction zone, to customary conditions for such selective hydrogenations and are set in accordance with the plant-specific boundary conditions and the purity to be achieved. For the selective hydrogenation of acetylene in C₂ streams, it is usual to set a space velocity of the gaseous C₂ stream of from 500 m³/m³*h, based on the catalyst volume, to 10 m³/m³*h at from 0° C. to 250° C. and a pressure of from 0.01 bar to 50 bar (in each case gauge pressure, bar g) and to add a total (i.e. over all reaction zones) of from 1 to 2 mol of hydrogen per mole of acetylene in the C₂ stream. For the selective hydrogenation of propyne and propadiene in C₃ streams, it is usual to set a space velocity of the gaseous C₃ stream of from 500 m³/m³*h, based on the catalyst volume, to 10 000 m³/m³*h at from 0° C. to 250° C. and a pressure of from 1 bar to 50 bar and to add a total of from 1 to 3 mol of hydrogen per mole of propyne and propadiene in the C₃ stream. For the selective hydrogenation of alkynes, dienes, alkenynes and/or polyenes in C₄ streams, it is usual to set a space velocity of the gaseous C₄ stream of from 200 m³/m³*h, based on the catalyst volume, to 10 000 m³/m³*h at from 0° C. to 300° C. and a pressure of from 1 bar to 30 bar and to add from 1 to 10 mol of hydrogen per mole of carbon-carbon multiple bonds to be hydrogenated in the alkynes, dienes, alkenynes and/or polyenes to be removed.

It is possible to add all the hydrogen upstream of the first reaction zone. However, the hydrogen is preferably introduced between the individual reaction zones in partial amounts calculated so that the desired degree of conversion of the alkynes, dienes, alkenynes and/or polyenes to be hydrogenated is in each case achieved in the next reaction zone but undesirable overhydrogenation of desired products to alkanes does not occur or occurs only to a tolerably small extent.

The overall conversion of alkynes, dienes, alkenynes and/or polyenes necessary over all reaction stages is determined by the residual amounts of these compounds which are tolerable in the selectively hydrogenated product stream, which are in turn determined by the further use to which the latter is to be put and are typically in the region of a few ppm by volume. An overall conversion of precisely 100% (i.e. a residual content of alkynes, dienes, alkenynes and/or polyenes of precisely 0 ppm by volume) is usually not set, since this would result in an undesirably high loss of desired product due to hydrogenation to the corresponding alkane. It is usual to operate the first reaction zone so that the major part of the overall conversion occurs there; a typical value is in the range from 60 to 70 mol % conversion, based on the alkynes, dienes, alkenynes and/or polyenes originally present. If only two reaction stages are used, a somewhat higher degree of conversion is usually set in the first reaction zone than is the case for a three-stage or multistage process. In a two-stage process, the residual conversion necessary to reach the desired maximum residual content of alkynes, dienes, alkenynes and/or polyenes is set in the second reaction zone. If the process is carried out in three stages, a typical degree of conversion in the second reaction zone is from 30 to 40 mol %, so that a total conversion of more than 90 mol % and up to almost 100 mol % is achieved at the outlet of the second reaction zone. In the third reactor, the desired residual conversion necessary to achieve removal of the alkynes, dienes, alkenynes and/or polyenes to the tolerable residual content is then set. If more than three reaction zones are used, the conversion is spread analogously over the reaction zones used. The conversion is, as is customary, set by appropriate setting of the process parameters such as temperature, space velocity, pressure or recycle ratio.

The content of the alkynes, dienes, alkenynes and/or polyenes to be removed from the feed stream to be hydrogenated selectively determines the number of reaction zones to be employed in a particular case. For example, at typical alkyne, diene and/or polyene contents, a C₂-hydrocarbon stream is treated in two reaction zones and a C₃-hydrocarbon stream is treated in two or three reaction zones. The other process conditions can also have an influence on the number of reaction zones, for example an isothermally operated reaction zone can replace two or more adiabatically operated reaction zones between which intermediate cooling would have to be provided to remove the heat of reaction.

In the process of the present invention, the hydrogen content upstream of the penultimate reaction zone and the conversion in the penultimate reaction zone are set so that the reaction mixture contains at least 0.7% by volume of hydrogen at the outlet of the penultimate reaction zone. This hydrogen content is preferably at least 0.8% by volume and particularly preferably at least 0.9% by volume. Furthermore, it is generally not more than 2% by volume, preferably not more than 1.8% by volume and particularly preferably not more than 1.6% by volume. Measures for setting a particular hydrogen content at the outlet of a reactor or a reaction zone are known. In particular, the temperature in this reaction zone can be reduced, for instance by appropriate cooling of the reactor in case of isothermal operation or of the feed to the reactor in the case of adiabatic operation, and/or the throughput through the reactor can be increased so that the hydrogen conversion in this reaction zone is not 100 mol % but drops to such a value that the desired hydrogen content is obtained at the outlet of this reaction zone. In addition, the excess of hydrogen upstream of this reaction zone can be made so high (by reducing the hydrogen conversion in one or more of the preceding reaction zones or by appropriate introduction of hydrogen upstream of the penultimate reaction zone) that the desired hydrogen content is obtained at the outlet of the penultimate reaction zone.

The further design of the plant for carrying out the process of the present invention, including the fixing of the number of reaction zones employed, the removal of heat, any recirculation loops and the selection of the temperatures, pressures, space velocities and other process parameters otherwise employed in operation, is carried out in the manner generally customary for such hydrogenation processes.

Catalysts used in the individual reaction zones are generally catalysts which are suitable for the hydrogenation of alkynes, dienes, alkenynes and/or polyenes in olefin-containing hydrocarbon streams. The use of highly selective catalysts (i.e. ones which preferentially hydrogenate alkynes, dienes, alkenynes and/or polyenes to olefins and hydrogenate olefins to alkanes to only a slight extent) is preferred. When less selective catalysts are used, it may even prove to be impossible to set the hydrogen content employed according to the present invention, namely when any hydrogen present reacts completely with the olefins which are naturally present in excess in such processes in the presence of these catalysts to form alkanes. In general, the process of the present invention can therefore be carried out using any catalyst for the hydrogenation of alkynes, dienes, alkenynes and/or polyenes which is sufficiently selective to allow the setting of the hydrogen content employed according to the present invention. This can, if necessary, be established in a routine test.

Known high-selectivity catalysts for the hydrogenation of alkynes, dienes, alkenynes and/or polyenes in olefin-containing hydrocarbon streams, which can also be used in the process of the present invention, typically comprise a metal of group 10 of the Periodic Table of the Elements (nickel, palladium, platinum) and optionally also a metal of group 11 of the Periodic Table of the Elements (copper, silver, gold) on a catalyst support. Use is often made of palladium catalysts or silver-doped palladium catalysts on a particulate oxidic support, frequently aluminum oxide. Such catalysts and their production are well known, cf., for example, EP-A-992 284 as cited at the outset and the documents cited therein, which are hereby expressly incorporated by reference.

In general, a catalyst comprising a metal of group 10 of the Periodic Table of the Elements on a catalyst support is used in at least one reaction zone. This catalyst may, if desired, further comprise an element of group 11 of the Periodic Table of the Elements. The metal of group 10 of the Periodic Table of the Elements present in the catalyst is preferably palladium and the metal of group 11 of the Periodic Table of the Elements is preferably silver. Preference is likewise given to the catalyst support being an oxidic catalyst support, for example aluminum oxide. In particular, such a catalyst is used in the penultimate reaction zone.

In the process of the present invention, particular preference is given to using a catalyst comprising a metal of group 10 of the Periodic Table of the Elements (nickel, palladium, platinum) and optionally a metal of group 11 (copper, silver, gold) of the Periodic Table of the Elements on a structured catalyst support or monolith made up of woven or knitted wire mesh, wire felt or foils or metal sheets, which may also be perforated, in at least the penultimate reaction zone. The catalyst preferably comprises palladium and optionally silver. Such catalysts and their production are likewise well known, for example from U.S. Pat. No. 5,866,734, EP-A-827,944 and EP-A-965,384 as cited at the outset and the documents cited in each of these, which are hereby expressly incorporated by reference.

### EXAMPLE

The second reactor of a customary plant for the hydrogenation of alkynes, dienes, alkenynes and/or polyenes in a propene-containing C₃ stream from a steam cracker, which comprised three adiabatically operated reactors connected in series, was equipped with a palladium/silver catalyst on a knitted wire mesh support in monolith form (produced as described in EP-A-965 384). The plant was operated in a normal manner at conventional parameters using a C₃ stream. After a running time of 90 days, the temperature of the second reactor was reduced from the customary value in the range from about 80 to 85° C. to a value in the region of 65° C., as a result of which the hydrogen content in the reaction mixture at the outlet of the second reactor increased to values in the range from 0.8 to 1.8% by volume. The other two stages continued to be operated using conventional catalysts and customary operating conditions.

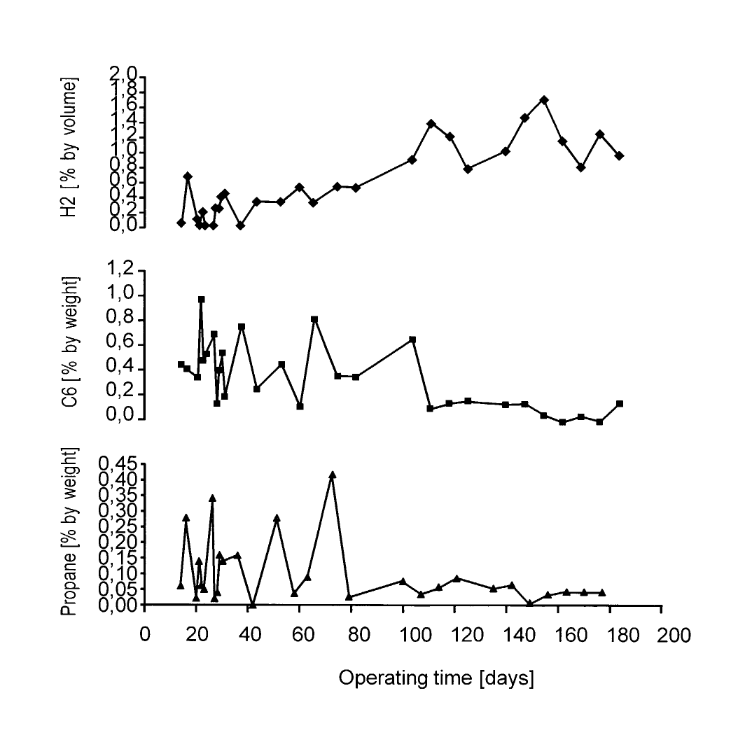

FIG. 1 shows the detailed results in graph form.

It was surprisingly found that despite this oversupply of hydrogen (H₂), the undesirable overhydrogenation to propane, i.e. the propane content of the output from the reactor, tended to decrease and stabilize at low values, and green oil formation, expressed as the content of C₆ compounds in the output from the reactor, decreased significantly.

We claim:

1. A process for the selective catalytic gas-phase hydrogenation of alkynes, dienes, alkenynes and/or polyenes in an olefin-containing hydrocarbon stream in at least two reaction zones connected in series without introduction of part of this hydrocarbon stream between the penultimate reaction zone and the last reaction zone, wherein the hydrogen content in the reaction mixture upstream of the penultimate reaction zone and the degree of conversion in the penultimate reaction zone are set so that the reaction mixture contains at least 0.7% by volume of hydrogen at the outlet of the penultimate reaction zone.

2. A process as claimed in claim 1, wherein acetylene in a C2 stream is hydrogenated.

3. A process as claimed in claim 1, wherein propyne and propadiene in a C3 stream are hydrogenated.

4. A process as claimed in claim 1, wherein butyne, but-3-en-1-yne, 1,2-butadiene and/or 1,3-butadiene in a C4 stream are hydrogenated.

5. A process as claimed in claim 1, wherein alkynes, dienes and/or polyenes in an olefin-containing hydrocarbon stream are hydrogenated in two or three reaction zones connected in series.

6. A process as claimed in claim 1, wherein catalysts comprising a metal of group 10 of the Periodic Table of the Elements on a catalyst support are used in the reaction zones.

7. A process as claimed in claim 6, wherein a catalyst comprising a metal of group 10 and a metal of group 11 on a catalyst support is used in at least one reaction zone.

8. A process as claimed in claim 6, wherein catalysts comprising palladium on a catalyst support are used.

9. A process as claimed in claim 7, wherein a catalyst comprising palladium and silver on a catalyst support is used in at least one reaction zone.

10. A process as claimed in claim 6, wherein a catalyst comprising a metal of group 10 and optionally a metal of group 11 on a structured catalyst support or monolith made up of woven wire mesh, knitted wire mesh, wire felt or foils or metal sheets, which may if desired be perforated.
